(19) United States
(12) Patent Application Publication
(10) Pub. No.: US 2013/0046299 A1
(43) Pub. Date: Feb. 21, 2013

(54) INTELLIGENT ELECTROSURGICAL ELECTRODE AND TRACKING SYSTEM

(71) Applicant: John J. Newkirk, Evergreen, CO (US)

(72) Inventor: John J. Newkirk, Evergreen, CO (US)

(21) Appl. No.: 13/585,014

(22) Filed: Aug. 14, 2012

Related U.S. Application Data
(60) Provisional application No. 61/525,134, filed on Aug. 18, 2011.
(60) Provisional application No. 61/594,087, filed on Feb. 2, 2012.

(51) Int. Cl. A61B 18/14

(52) U.S. Cl. 606/41

(57) ABSTRACT

An intelligent electrode device for monitoring use of an electrosurgical electrode. An example intelligent electrode includes an electrosurgical electrode (monopolar or bipolar), and a tracking element to communicate electrode parameters to an RF generator or other controller and track the use of the electrosurgical electrode. A shroud houses the electrosurgical electrode and the tracking element. A mechanical interconnect attaches the shroud to a pen. An electrical connector provides electrical power from the pen to the electrosurgical electrode.

### PRIORITY CLAIM

This application claims priority to U.S. Provisional Patent Application No. 61/525,134 titled “Traceable Electrode And Tracking System For Single-Use Surgical Needles” of John J. Newkirk filed on Aug. 18, 2011, and U.S. Provisional Patent Application No. 61/594,087 titled “Handheld Electrosurgical Generator” of John J. Newkirk filed on Feb. 2, 2012, each of which is hereby incorporated by reference as though fully set forth herein.

### BACKGROUND

Electrosurgery is a form of surgery in which body tissue is cut or cauterized by a high frequency current, and offers certain advantages to conventional knife dissection. A variety of electrosurgical tools have been employed. The electrosurgical electrode has been used for many years in a wide variety of applications, e.g., for delicate cutting, cauterization, and normal surgical cutting as with a scalpel. When in a cutting mode, the current created when the electrosurgical electrode touches the body tissue incises the tissue. By varying the mode of the RF generator output, it is also possible to utilize the electrosurgical device to enhance cauterization or coagulation of blood in a wound. In the cauterizing mode, the electrosurgical electrode generates much more heat than when in the cutting mode.

### BRIEF DESCRIPTION OF THE DRAWINGS

FIG. 1a is a perspective view of an example intelligent electrode for electrosurgical electrodes.

FIG. 1b is a perspective view of the example intelligent electrode shown in FIG. 1a with a shroud shown removed from a base portion,

FIGS. 2a-c are top plan views of the example intelligent electrode shown in FIG. 1a, illustrating a connection to an electrosurgical pen.

FIG. 3a is a side plan view of the example intelligent electrode shown in FIG. 1a.

FIG. 3b is a cross-sectional view showing the interior of the example intelligent electrode corresponding to the side plan view shown in FIG. 3a.

FIG. 4a is an enlarged view of another example intelligent electrode shown in an unassembled configuration.

FIG. 4b is an enlarged view of the example intelligent electrode in FIG. 4a shown in an assembled configuration.

FIG. 5a is a front plan view of an example electrical/mechanical interconnect for the example intelligent electrode shown in FIG. 4a.

FIG. 5b is a side view of the example electrical/mechanical interconnect shown in FIG. 5a.

FIG. 5c-d are enlarged views of an electrical connector for the example intelligent electrode shown in FIG. 4a, wherein c) shows the electrical connector in a disconnected configuration, and d) shows the electrical connector in a connected configuration.

FIG. 6 is a schematic diagram of an example microchip which may be used in the example intelligent electrode shown in FIG. 1a or 4a,

FIG. 7 is a high-level diagram illustrating the example intelligent electrode shown in either FIG. 1a or 4a implemented as part of a tracking system for electrosurgical electrodes.

FIG. 8 is a flowchart illustrating example operations, which may be implemented to monitor an electrosurgical electrode.

### DETAILED DESCRIPTION

Electrosurgical electrodes may be composed of stainless steel, although some are composed of other alloys such as those containing primarily tungsten, molybdenum, chromium, nickel or cobalt. More recent electrodes implement an ultra-sharp refractory alloy. Such ultra-sharp electrodes (also known as “electrosurgery needles”) have been specifically designed for consistency, symmetry, sharpness and tapering to produce superior results. For example, use of an ultra-sharp needle allows for reduced RF power due to the concentration of energy at the ultra-sharp needle tip. Efficient cutting at lower power reduces blood loss and leads to much cleaner and less traumatic cuts, resulting in less scar tissue. Use of the ultra-sharp needle also eliminates the drag when cutting tissue. This “no-touch” technique allows the surgeon a sensitive “feel”, which is a significant benefit when performing extreme microsurgery. When used in the cauterization mode, the ultra-sharp electrode may again be used at relatively lower RF power, thereby eliminating problems associated with excessive heat, such as melting the electrode needle, and with greater control over the direction and location of sparking.

Several electrode configurations or circuit topologies may be used in electrosurgery. In a “monopolar” configuration, the patient is attached to the return electrode via a relatively large metal plate or a flexible metalized plastic pad which is connected to the return electrode of the RF generator. The surgeon uses an electrosurgical electrode or “needle” to make contact with the tissue. In a “bipolar” configuration, the voltage is applied to the patient using a pair of similarly sized electrodes or needles. For example, special forceps may be used, e.g., with one tine connected to one pole of the RF generator and the other tine connected to the other pole of the generator. When a piece of tissue is held by the forceps, RF current flows from one to the other forceps tine, heating the intervening tissue.

It is noted that as used herein, the terms “electrosurgery needle,” “electrosurgical needle,” “electrosurgical electrode,” and “electrode” apply to, but are not limited to, both monopolar and bipolar configurations.

An electrode's geometry, material, sharpness, and other characteristics can affect variables controlled by the RF generator to achieve efficient cutting, heating, and/or coagulation. For example, an ultra-sharp tip may use a different power setting and/or waveform than a flat or rounded stainless steel tip.

Electrosurgery needles give the surgeon unprecedented precision during surgery, resulting in the patient experiencing less bleeding, less scarring, and faster healing than standard methods of surgery. Although the cost of electrosurgery needle may at first seem high, the needle actually represents one of the lowest costs of surgical procedure. A recent study comparing laser surgery with electrosurgery revealed significant reductions in time, blood loss, and postoperative pain over the course of 20 typical bilateral reduction mammoplastys. See, e.g., Table 1:

TABLE 1
Laser versus Electrocautery

| | |
|---|---|
| Laser | Electrocautery |

| | | | | |
|---|---|---|---|---|
| Dissection Time | 61 | minutes | 32 | minutes |
| Blood loss | 176 | cc | 72 | cc |

| | | |
|---|---|---|
| Est. Cost | $698 | $114 |

Source: Michigan State University

Electrosurgery needles are typically manufactured as limited-use or even “single-use” needles. The term “single-use” is not intended to encompass a single cut, but generally a single surgery (which may involve multiple cuts). Considerations as to what may constitute a single use include, but are not limited to, the duration of use, electrical power being used, type of surface being operated on, and extent and types of cuts being made. As such, more than one “single-use” electrosurgery needle may need to be used during a single, yet extensive surgery.

In any event, marking the electrosurgery needles as “single-use” does not stop customers (e.g., surgeons or surgical technicians) from reusing the needles. Often, the customer believes that simply sterilizing (e.g., by autoclaving) the electrosurgery needles is sufficient and that the electrosurgery needles can then be safely reused. As such electrosurgery needles intended to be limited-use or single-use needles are often reused to the point of being damaged, which can produce undesired results and even harm the patient.

There are several reasons for the limited-use or single-use designation, and manufacturers typically do not recommend the reuse of electrosurgery needles. The tungsten tip of an ultra-sharp electrosurgery needle is very delicate, and although the needle generally does not melt during use, the needle can be damaged with repeated use. The ultra-sharp point, on which the superior performance of the electrosurgery needle depends, is vulnerable to mechanical distortion, requiring that the tip be handled with great care. Example distortion includes barbing or blunting which can occur with multiple use or rough handling.

In addition, each electrosurgery needle is typically shipped with a protective rubber ring over the sharp end. This ring protects the tip during initial autoclaving and other handling until it is used on the patient. Autoclaving the electrosurgical electrode beyond an initial sterilization (e.g., multiple times for repeated use) may also weaken the bond between the insulation and the electrosurgery needle body.

Further, while forceps should not be used to grasp the electrosurgery needle, electrosurgery needles are often returned to the manufacturer as “defective” which exhibit rips and tears on the insulation indicating hemostatic forceps have been used. In extreme cases, multiple autoclavings combined with rough treatment may result in punctures to the insulation, which in turn may result in the patient being burned or otherwise injured during a surgical procedure. In an example where electrosurgery needles were reused over a period of several months, the ultra-sharp tips are often dulled and/or the insulation is often damaged.

An intelligent electrode and tracking system for electrosurgical electrodes is disclosed, which enable automatic identification of needle type and characteristics, monitoring and/or tracking use of the needles, and/or providing data including evidence of reuse to a manufacturer and/or alerting the user that the electrosurgical electrode has been re-used or is approaching (or has surpassed) the recommended lifetime of the needle.

In an example, the intelligent electrode may be designed for ease-of-use, and easily implemented by a surgeon or other end-user in his or her daily practice with limited or even no understanding of electronics, software, and the operations described herein. For example, a surgeon or other end-user may simply “plug-and-play” a wide variety of electrode types (e.g., both cutting and cauterizing) by inserting the intelligent electrode into a handle or pen, using it for a surgery, and then discarding the intelligent electrode and using a new one for the next procedure. Special knowledge of each electrode's RF characteristics is not needed. The intelligent electrode can communicate such parameters automatically and directly with the RF generator. Feedback provided to the surgeon or other end-user, if any, is simple to understand and utilizes non-intrusive technologies such as tablet devices and RF generators already in use in many operating rooms in the United States.

An example of the intelligent electrode and tracking system disclosed herein includes a shroud with connecting mechanism for attaching to an electrosurgical “pen” (also referred to herein as a “pen”). Power is provided from an RF generator to the needle via the pen. The pen can also be used as a handle for operating the needle during a surgical procedure. The intelligent electrode and tracking system includes a microchip in the shroud of the needle. The microchip may receive an electrical signal when power is provided by the RF generator to the needle. Thus, the microchip can record data related to time(s) of use, power output, duration of use, and even date/time of use, to name only a few examples of data types, so that use of the needle can be monitored or tracked/traced. The intelligent electrode may provide feedback to the RF generator (or other control circuitry) such that the generator can sense the type of electrode in use, and make adjustments and set parameters or control variables automatically. The intelligent electrode and tracking system may also provide feedback to the user, manufacturer, and/or monitoring service.

Before continuing, it is noted that as used herein, the terms “includes” and “including” mean but is not limited to, includes or “including” and “includes at least” or “including at least.” The term “based on” means “based on” and “based at least in part on.”

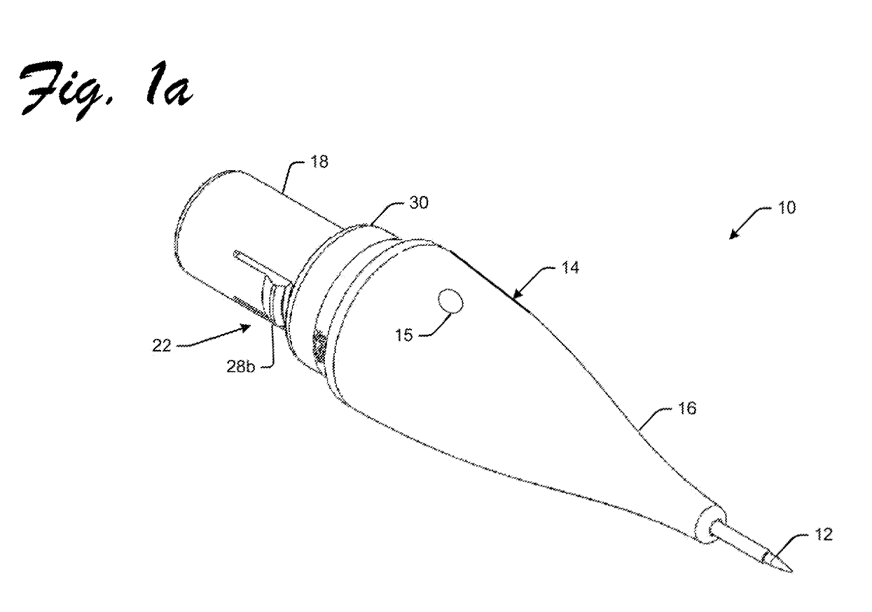

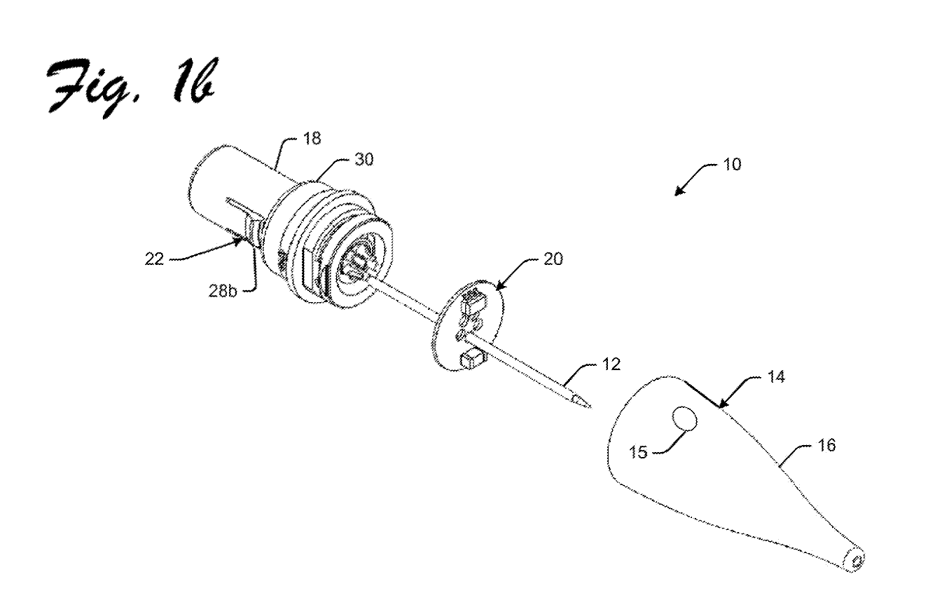

FIG. 1a is a perspective view of an example intelligent electrode 10 for electrosurgical electrodes 12, FIG. 1b is a perspective view of the example intelligent electrode 10 shown in FIG. 1a with a shroud 14 having a top portion 16 removed from a base portion 18. The intelligent electrode device 10 may be used for tracking and monitoring the electrosurgical electrode 12, providing feedback, and further enhanced functions which will be apparent from the disclosure herein. In an example, the electrosurgical electrode 12 may have a tungsten electrode tip with a radius of about 50 microns or smaller. However, the intelligent electrode device 10 may be used with any suitable needle.

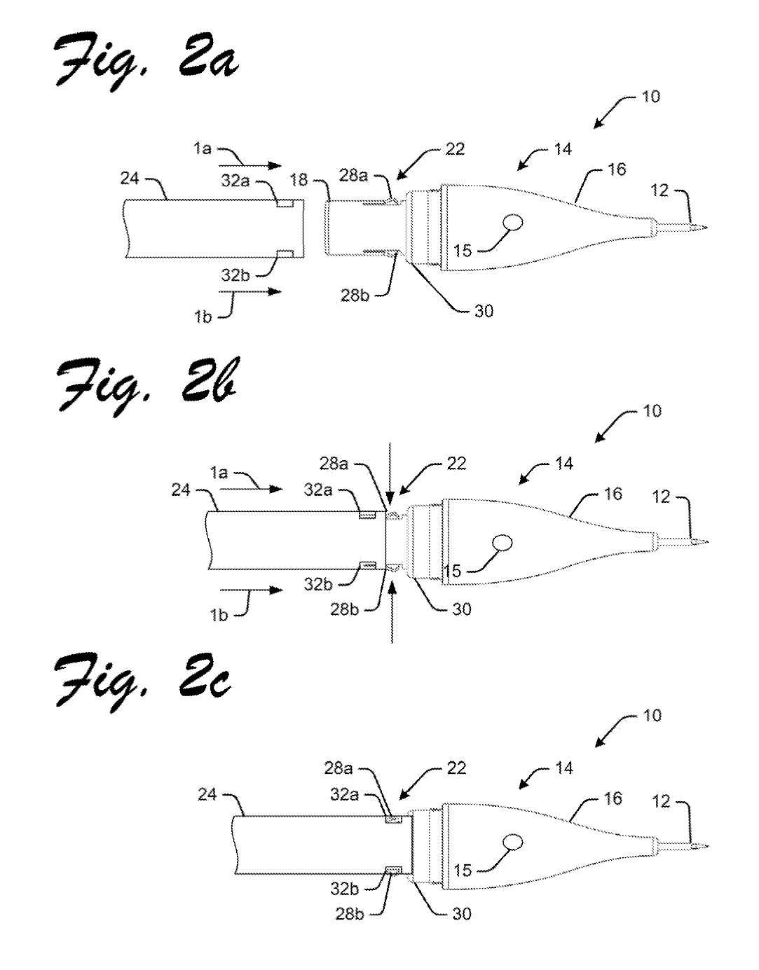

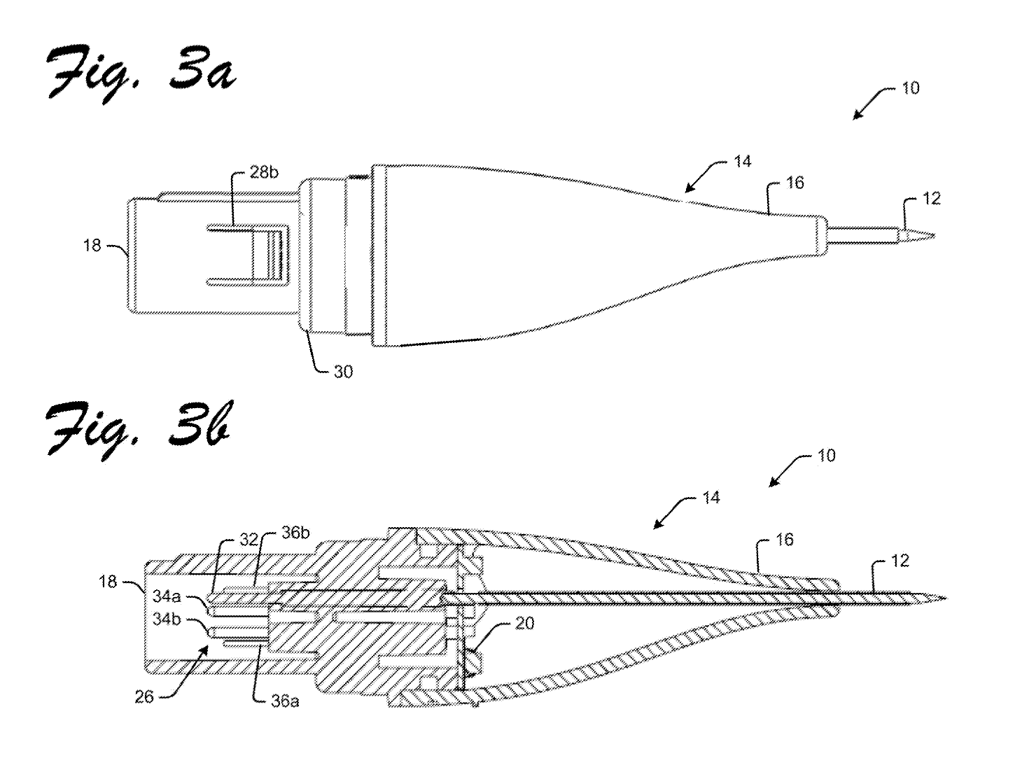

The intelligent electrode device 10 may include the electrosurgical electrode 12, and a tracking element 20 to indicate use of the electrosurgical electrode 12. The shroud 14 houses the electrosurgical electrode 12 and the tracking element 20. A mechanical interconnect 22 attaches the shroud 14 to a handle or “pen” 24 (seen in the illustration in FIGS. 2a-c). An electrical connector 26 (seen best in the cross-sectional view in FIG. 3b) provides electrical power from the pen 24 to the electrosurgical electrode 12 during use, and/or a data connection for the tracking element 20.

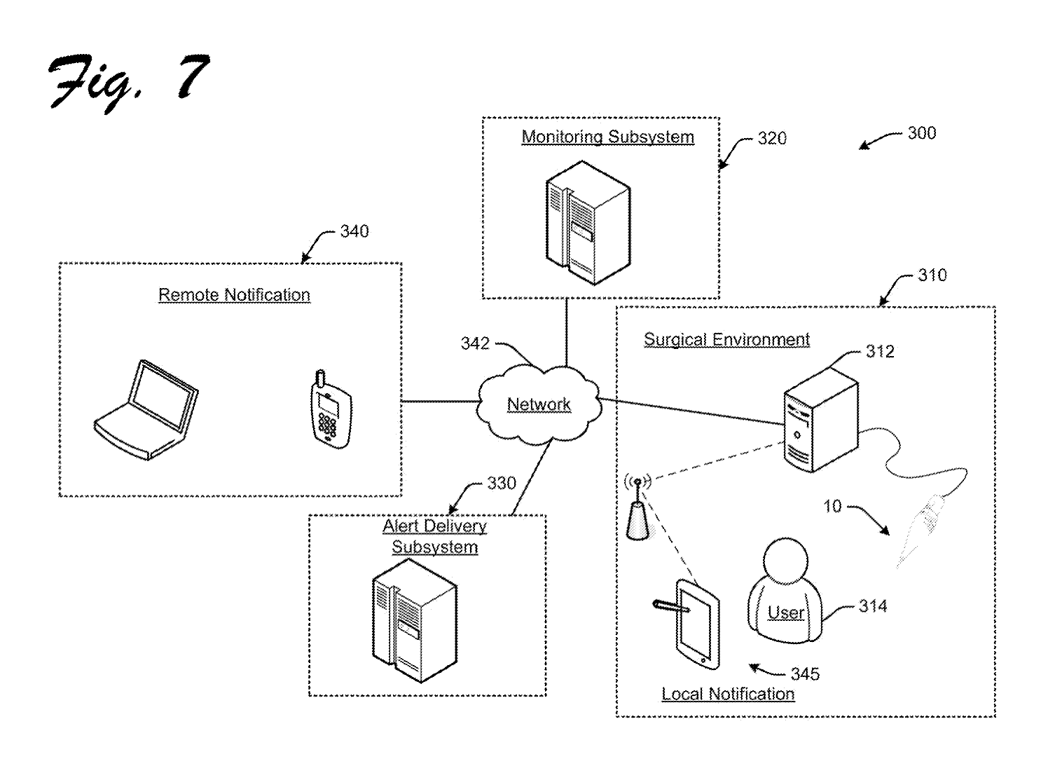

In an example, the tracking element 20 includes a microchip configured to at least store data from the tracking element 20 indicating, for example, the type of use, and other use characteristics (e.g., duration and/or number of times of use) of the electrosurgical electrode. The microchip may be a serial EEPROM (e.g., as shown in FIG. 6), which receives power when electrical power is delivered to the needle (i.e., when the electrosurgical electrode 12 is being powered, indicating use). Accordingly, the microchip may record actual use data corresponding to usage of the needle. In an example, the microchip may record this data and issue a data signal to an external processor for further processing. Such an embodiment, utilizing an external processor, enables the microchip embedded on the needle itself to be relatively small in size, while still providing use information related to the needle. However, the intelligent electrode 10 is not limited to use with an external processor, and may include at least some degree of processing itself, e.g., as part of the microchip.

Processing may include comparing use data to a threshold and issuing alerts when the threshold is satisfied and/or exceeded. Processing may also include correlating data for the electrosurgical electrode 12, e.g., to a serial number or other identifying indicia for the electrosurgical electrode 12. For example, different needles may have different use thresholds which are satisfied before an alert is issued.

The intelligent electrode 10 may use the data from tracking element 20 (and/or further processed data) to provide feedback to the user, manufacturer, and/or monitoring service. For example, the intelligent electrode 10 may be used with a tracking system to issue alerts to prevent or reduce reuse or overuse of the electrosurgical electrode 12.

It is noted that the electrosurgical electrode 12 shown and described herein is an example of a suitable monopolar electrode which may be used, and is illustrative, but not intended to be limiting. Other monopolar electrodes, bipolar electrodes, and other electrodes now known or later developed may also be used, as will be readily understood by those having ordinary skill in the art after becoming familiar with the teachings here. For example, the electrosurgical electrode 12 could be a pair of similarly-sized electrodes. Other electrode configurations are also possible.

It is also noted that the intelligent electrode 10 may be used for type determination and “parameter preset” aspects of the needle, along with traceability and other advanced features. By using the intelligent electrode 10, the RF generator or other controller (e.g., processor and/or control circuitry) immediately recognized the type of needle being used, and can preset parameters accordingly (e.g., bipolar or monopolar, maximum power, minimum power, waveform type) on the RF generator. This is a valuable tool for surgeons, i.e., to be able to “plug-and-play” without having to manipulate numerous RF generator controls, as the intelligent electrode 10 removes most if not all of the guesswork. In general, surgeons do not care about the technical aspects of the RF generator (e.g., whether a sine wave vs. sawtooth vs. blend is being deployed). The surgeon just wants to snap in the electrode and get to work, controlling mainly the RF output power based on tissue type being encountered during the surgery.

FIGS. 2a-c are top plan views of the example intelligent electrode 10 shown in FIG. 1a, illustrating a connection to an electrosurgical pen 24 (also referred to herein as a “pen”). In FIG. 2a, the shroud 14 is shown having tabs 28a-b on the base portion 18. While the tabs 28a-b may be made of metal and separately attached to the base portion, the tabs 28a-b may also be manufactured as part of the base portion, e.g., as plastic.

The tabs 28a-b are in a default position in FIG. 2a. In FIG. 2b, as the base portion 18 is inserted into the pen 24 (e.g., by press fitting illustrated by arrows 101a-b), the pen 24 depresses the tabs 28a-b so that the pen 24 fits over the tabs 28a-b and can be pushed up to the collar 30 of the shroud 14. When the pen 24 is pressed all the way up to the collar 30 as shown in FIG. 2c, the tabs 28a-b pop out inside openings 32a-b formed in the pen 24 to secure the intelligent electrode 10 to the pen 24.

To remove the pen 24 from the intelligent electrode 10, the tabs 28a-b may be depressed (e.g., by squeezing between the user's thumb and forefinger) or “snapped” out via a friction fit by applying an extraction force. The tabs 28a-b release from the openings 32a-b formed in the pen 24 so that the intelligent electrode 10 may be withdrawn from the pen 24 by pulling on the intelligent electrode 10.

As such, the intelligent electrode 10 may be disposable when it has met the use threshold. In another example, the intelligent electrode 10 can be recycled by returning the intelligent electrode 10 to the manufacturer to replace the electrosurgical electrode 12 and reset or replace the tracking element 20. But in either case, the user does not need to replace the pen 24.

FIG. 3a is a side plan view of the example intelligent electrode 10 shown in FIG. 1a. FIG. 3b is a cross-sectional view showing the interior of the example intelligent electrode corresponding to the side plan view shown in FIG. 3a, The electrosurgical electrode 12 can be seen housed in the shroud 14. In the cross-sectional view, the electrosurgical electrode 12 is shown connected to contact 32 of the electrical connector 26. Power may be provided to the electrosurgical electrode 12 through the pen 24 via contact 32 of the electrical connector 26 (e.g., from an RF generator, not shown). That is, the electrical connector 26 may be connected to a mating connection in the pen 24 when the pen 24 is connected to the intelligent electrode 10 (e.g., as described above with reference to the illustration shown in FIGS. 2a-c).

In an example, power may be provided from an RF generator through the pen 14 via the electrical connector 26 in the intelligent electrode 10. The tracking element may monitor or track use of the electrosurgical electrode 12. For example, the tracking element 20 may be powered on by providing power to the electrosurgical electrode 12, via the same contact 32 or separate contact. A microchip and/or associated circuitry in the tracking element 20 may track use based on time and/or power being provided to the electrosurgical electrode 12.

The electrical connector 26 may also include a data connection for the tracking element 20. For example, the electrical connector 26 may include an input line 34a, an output line 34b, a power line 36a, and a ground line 36b. Other configurations are also possible, including hardwired and wireless data connections. The data connection enables use data from the tracking element 20 to be shared externally. For example, the data connection may be established with output or feedback device(s) (not shown) in the pen 24 or other instrumentation. The data connection may also be established with the user's personal computer, or used by the manufacturer to read-out data from the tracking device 20 (e.g., to monitor compliance with the limited-use designation and warranty/replacement provisions).

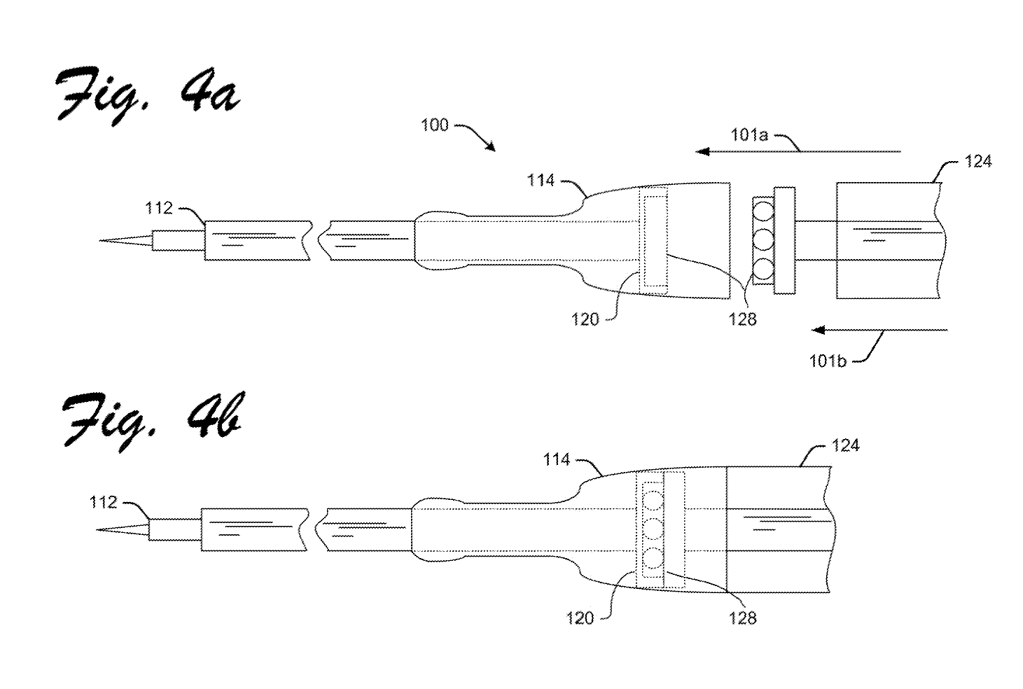

FIG. 4a is an enlarged view of another example intelligent electrode 100 shown in an unassembled configuration. FIG. 4b is an enlarged view of the example intelligent electrode 100 in FIG. 4a shown in an assembled configuration. The example intelligent electrode 100 includes an electrosurgical electrode 112 connected to a shroud 114 with an electric-mechanical interconnect 128 for attaching the needle to a handle or pen 124 (e.g., by press fitting illustrated by arrows 101a-b).

Power may be provided to the electrosurgical electrode 112 via the pen 124 (e.g., from an RF generator) as described above. The mechanical connection of the electric-mechanical interconnect 128 is used to attach the intelligent electrode 100 to the pen 124. The electric-mechanical interconnect 128 also includes electrical contacts to provide a power and data connection with the pen 124. As discussed above, the electrical contacts may include an input line, an output line, a power line, and a ground line, although other configurations are also possible. The intelligent electrode 100 also includes a tracking element (such as a microchip and associated circuitry) in the shroud 114. The microchip may be embedded in the shroud 114 so that it cannot be readily removed.

In an example, the tracking element 120 may include a microchip such as a serial EEPROM (e.g., as shown in FIG. 6), which receives power when electrical power is delivered to the electrosurgical electrode 112 (i.e., the needle is used). Accordingly, the microchip may record actual use data corresponding to usage of the needle. The microchip may record this data and issue a data signal to an external processor for further processing. Such an embodiment, utilizing an external processor, enables the microchip embedded on the needle itself to be relatively small in size, while still providing use information related to the needle.

Further processing may include comparing use data to a threshold and issuing alerts when the threshold for the electrosurgical electrode 112 is satisfied and/or exceeded. Further processing may also include correlating the data for the electrosurgical electrode 112, e.g., to a serial number or other identifying indicia for the needle. For example, different needles may have different use thresholds which are satisfied before an alert is issued.

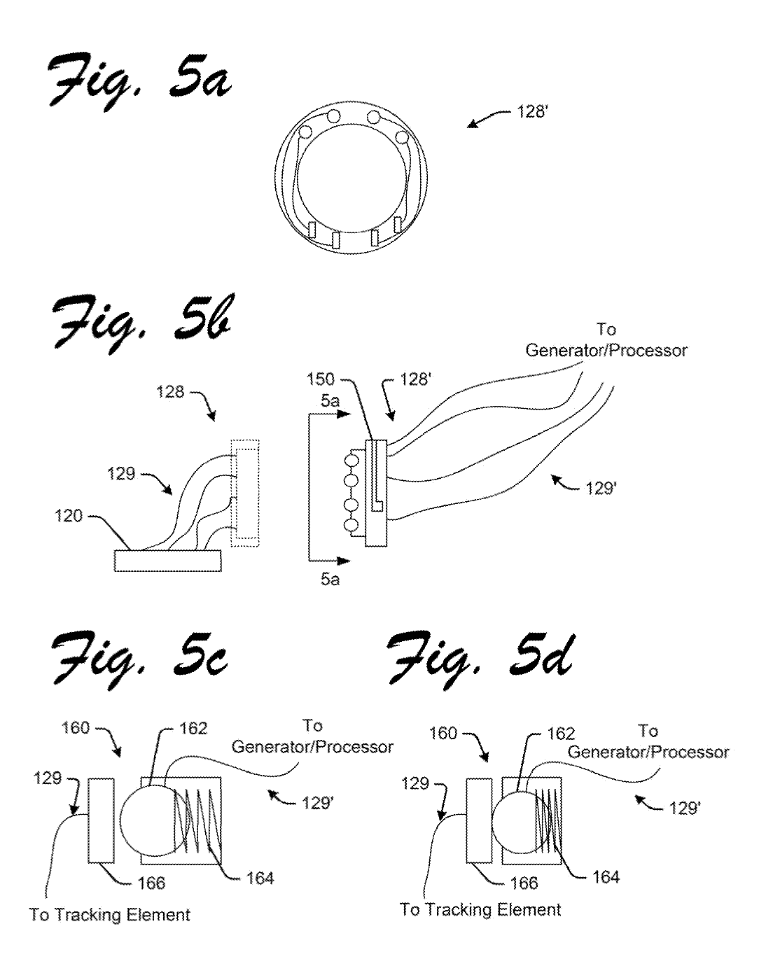

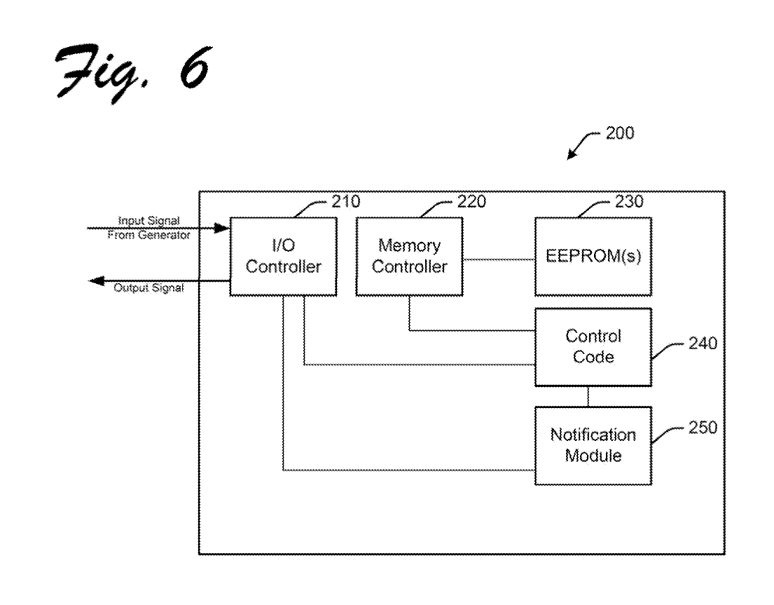

FIG. 5a is a front plan view of an example electrical/mechanical interconnect 128′ (as viewed in the direction of lines 5a-5a in FIG. 5b) for the example intelligent electrode 100 shown in FIG. 4a. FIG. 5b is a side view of the example electrical/mechanical interconnect 128 shown in FIG. 5a, showing both the connector 128 on the shroud 114 and the mating connector on the pen portion 128′. Wiring 129 and 129′ is also illustrated, including data (I/O), power and ground lines between the generator and/or external processor (not shown) and the tracking element 120. A rotatable interlock mechanism 150 is also illustrated as it may be rotated into place and connected to mechanically secure the two portions of the electrical/mechanical interconnect 126 together (e.g., as can be seen in FIG. 4b).

FIG. 5c-d are enlarged views of an electrical connector 160 for the example intelligent electrode 100 shown in FIG. 4a, wherein c) shows the electrical connector 160 in a disconnected configuration, and d) shows the electrical connector 160 in a connected configuration. It can be seen that individual ball bearings 162 (e.g., one for each data and power connection) may be outwardly biased (e.g., by spring 164). When the pen 124 is inserted into the shroud 114 and rotated into place, the ball bearings 162 push against the biasing mechanism (e.g., spring 164) and “give” inwardly (e.g., toward the pen 124) to enable the mechanical connection while establishing a good electrical connector between the ball bearing 162 in the pen 124 and the contact pads 166 in the shroud 114.

It is noted that the electricemechanical interconnect 128 shown and described herein is an example of a suitable interconnect which may be used, and is illustrative, but not intended to be limiting. Other interconnects now known or later developed may also be used, as will be readily understood by those having ordinary skill in the art after becoming familiar with the teachings here. For example, the electrical/mechanical interconnect 128 does not need to include ball bearings and pads. Other contacts are also possible.

FIG. 6 is a schematic diagram of an example tracking element 200 which may be used as part of the tracking element 20 and 120 in the example intelligent electrodes 10 and 100 shown in FIG. 1a or 4a, respectively. For purposes of illustration, the tracking element 200 may be a microchip and associated circuitry, and includes an input/output controller 210, memory controller, 220, EEPROM(s) 230. The tracking element 200 may be operated to execute control code 240 (e.g., software or firmware described below) to drive a notification module 250.

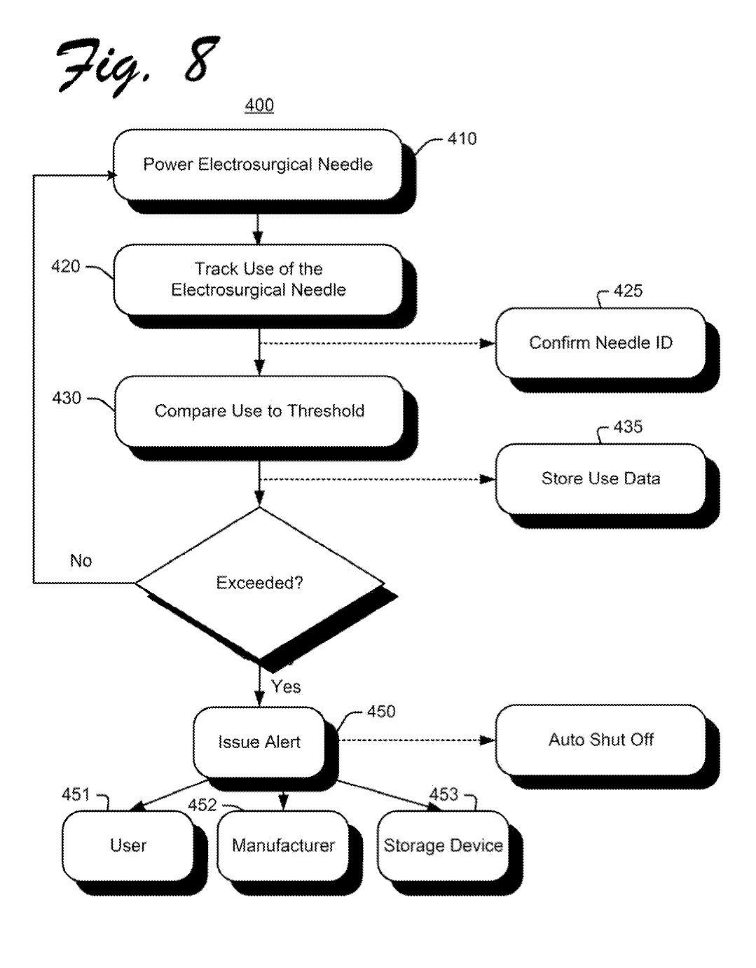

The tracking element 200 may receive input when power is delivered to the electrosurgical electrode. Accordingly, the tracking element 200 may be used to record time of first use, power output, time of use, and date, to name only a few examples of data types, so that the needle can be monitored or tracked. The tracking element 200 may also deliver an output signal including recorded data to an alert delivery subsystem, the RF generator (other external processor), and/or to a user feedback device in the shroud of the intelligent electrode itself. For example, the output signal may, also be delivered as a local alert, such as a visual display on a usage meter on the electrosurgical electrode itself or to a tablet or other computing device (e.g., as shown in FIG. 7). Other examples may also include audible and/or tactile feedback.

The intelligent electrode 10 or 100 may also be implemented as part of or with a tracking system to provide feedback to the user and/or the manufacturer, issuing alerts when a needle is used more than recommended. For example, a user may be notified when a single-use needle has been used more than once.

FIG. 7 is a high-level diagram illustrating the example intelligent electrode 10 (or 100 shown in FIG. 4a), implemented as part of a tracking system 300 for electrosurgical electrodes. The intelligent electrode 10 may be used in a surgical environment 310, including the intelligent electrode 10 connected to a generator 312 for use by a surgeon or other end-user 314. The end-user may be a separate person (e.g., a surgical technician assisting the surgeon) from the person using the intelligent electrode 10.

The tracking system 300 may include and/or be operatively associated with a monitoring subsystem 320 and an alert delivery subsystem 330 for automatically monitoring and notifying a user and/or manufacturer of repeated use of the electrosurgical electrode. The function of the monitoring subsystem 320 and the alert delivery subsystem 330 may be local (e.g., in the intelligent electrode device 10 and/or at the RF generator 312) and/or partially or wholly remote (e.g., offsite from the surgical environment, such as at a manufacturer's facility). In addition, notification may be provided remotely 340 (e.g., via a network 342) for the user and/or manufacturer and/or locally 345 to the surgical environment 310.

Notification may be based on a needle usage threshold (e.g., programmed into the microchip and/or monitored by the monitoring subsystem). The threshold may be “single-use” or other suitable threshold, e.g., based on duration of use and power delivery during use. It is noted that some needles may be used more than once before triggering the threshold. The threshold may be based on design considerations, such as but not limited to properties of the electrosurgical electrode 12 and/or the intended use.

The alert delivery subsystem 310 may issue an alert or notification via a feedback device 15. For purposes of illustration, feedback device 15 is illustrated in FIG. 1a as it may be provided in the shroud 14 of the intelligent electrode device 10, although any suitable feedback device may be provided at any desired location. In an example, the feedback device 15 may be an LED light that turns on or changes color based on use of the electrosurgical electrode 12 (e.g., from green to yellow to red as use progresses). In another example, the feedback device 15 may be a small speaker to emit an audible sound indicating excessive use or reuse of the electrosurgical electrode 12. In another example, the feedback device 15 may include a vibrating assemble to provide tactile feedback to the user.

In another example, the alert delivery subsystem 310 may issue an alert or notification by changing color of a portion of the needle. For example, the tracking element (e.g., microchip 200 shown in FIG. 6) may be connected to deliver an output signal to a color meter which includes a chemical substance that changes color in response to an electrical signal. The color of the colored substance may change based on the input signal, e.g., from green to yellow to red. In another example, the alert delivery subsystem 310 includes a sound issued in a vicinity of the electrosurgical electrode after repeated use of the electrosurgical electrode. Other feedback may be issued by the generator based on output received from the microchip and processing of that output at the generator (or other external processing device).

In another example, the alert delivery subsystem 310 may issue an email notification, e.g., issued by, a monitoring service. The email may be delivered to the user and/or manufacturer. Still other examples of an alert delivery subsystem 310 are also contemplated, as will be understood by those having ordinary skill in the art after becoming familiar with the teachings herein.

Before continuing, it should be noted that the examples described above are provided for purposes of illustration, and are not intended to be limiting. Other devices and/or device configurations may be utilized to carry out the operations described herein.

FIG. 8 is a flowchart illustrating example operations which may be implemented to monitor an electrosurgical electrode. Operations 400 may be embodied as program code or machine readable instructions (such as but not limited to software or firmware). The machine-readable instructions may be stored on a non-transient computer readable medium and are executable by one or more processor to perform the operations described herein. When executed on a processor, the logic instructions cause a general purpose computing device to be programmed as a special-purpose machine that implements the described operations. In an example, the components and connections depicted in the figures described above may be used.

Operation 410 includes powering the electrosurgical electrode. For example, power may be provided from an RF generator through the pen via an electrical connector in the intelligent electrode. Operation 420 includes tracking use of the electrosurgical electrode. For example, the tracking element may be powered on simultaneously with providing power to the electrosurgical electrode. A microchip and/or associated circuitry may track use based on time and/or power being provided to the electrosurgical electrode.

In an example, the needle identification may be confirmed in operation 425. A mismatch between an expected needle identification and the detected needle identification may indicate the needle has been replaced without authorization (e.g., by someone other than the manufacturer). Power may be terminated to the electrosurgical electrode (or other alert issued) if the needle identification does not match the needle identification accessed by the microchip. Operating parameters for the electrosurgical generator may also be preset based on the needle identification operation 425.

Operation 430 includes analysis of the data, for example, comparing use monitored by the tracking element to a threshold value. As noted above, the thresholds may be configured based on design considerations. The data obtained by the tracking device may also be stored in operation 435 (e.g., in memory in the intelligent electrode and/or externally). In operation 440, a determination is made whether use of the electrosurgical electrode has been (or will be) exceeding the recommended use (e.g., reuse of a single-use needle). If the electrosurgical electrode can continue to be used, tracking use continues in operation 420.

If use of the electrosurgical electrode exceeds the recommended use (e.g., based on the threshold compared in operation 440), an alert may be issued in operation 450. Issuing an alert may include, but is not limited to, issuing an alert to the user 451 of the intelligent electrode device (e.g., via audible, visual, and/or tactile output at or near the intelligent electrode device itself), to the manufacturer 452 (e.g., via a monitoring service), and/or to onboard and/or external storage 453 for later retrieval (e.g., read out from memory when the needle is returned to the manufacturer for replacement under warranty).

In an example, operation 455 may implement an automatic shut-off feature. The automatic shut-off feature may disable power to the electrosurgical electrode when the needle identification does not match the needle identification accessed by the microchip, as noted above during operation 425, and/or when the monitored use of the electrosurgical electrode exceeds the recommended use (e.g., based on the threshold compared in operation 440). It is noted that if the power is going to he automatically shut-off, it may be desirable to first issue at least one, if not multiple alerts to the user so that power to the electrosurgical electrode is not terminated at a critical point during a surgery. In another example, the power may remain on, but be terminated during a restart, wherein the restart indicates that the user is attempting to reuse the electrosurgical electrode,

The operations shown and described herein are provided to illustrate example implementations. It is noted that the operations are not limited to the ordering shown. Still other operations may also be implemented.

The operations may be implemented at least in part using a device, such as the intelligent electrode device and/or tracking system described above. In an example, the user is able to make predetermined selections, and the operations described above are implemented transparently to the user to monitor use of the electrosurgical electrode. It is also noted that various operations described herein may be automated or partially automated.

It is noted that the examples shown and described are provided for purposes of illustration and are not intended to be limiting. Still other examples are also contemplated.

1. An intelligent electrode device for determining type of and monitoring use of an electrosurgical electrode, the intelligent electrode comprising:
an electrosurgical electrode:
a tracking element to indicate type and usage of the electrosurgical electrode;
a shroud housing the electrosurgical electrode and the tracking element;
a mechanical interconnect to attach the shroud to a pen; and
an electrical connector to provide electrical power from the pen to the electrosurgical electrode.

2. The intelligent electrode device of claim 1 wherein the tracking element includes at least a microchip configured to at least store data from the tracking element indicating type and usage of the electrosurgical electrode.

3. The intelligent electrode device of claim 2 wherein the data from the tracking element include at least type of needle in use, time and date of first use, power output, date of manufacture, serial number, and total time of use.

4. The intelligent electrode device of claim 3 wherein the data indicate overuse of the electrosurgical electrode when a threshold is exceeded for time of use, power output, number of times used, and product expiration date.

5. The intelligent electrode device of claim 1, further comprising a monitoring subsystem to analyze use of the electrosurgical electrode indicated by the tracking element.

6. The intelligent electrode device of claim 1, further comprising a data connection to provide data indicating use of the electrosurgical electrode from the tracking element to a monitoring subsystem.

7. The intelligent electrode device of claim 6, wherein the data connection provides data indicating use of the electrosurgical electrode through the pen.

8. The intelligent electrode device of claim 6, wherein an alert delivery subsystem operatively associated with the monitoring subsystem issues an alert based at least in part on use of the electrosurgical electrode indicated by the tracking element.

9. The intelligent electrode device of claim 8, wherein the alert delivery subsystem issues an alert when the electrosurgical electrode is re-used.

10. The intelligent electrode device of claim 1, further comprising a user feedback device in the shroud.

11. The intelligent electrode device of claim 9, wherein the user feedback device changes color based at least in part on use of the electrosurgical electrode indicated by the tracking element.

12. The intelligent electrode device of claim g, wherein the user feedback device issues a sound In a vicinity of the electrosurgical electrode based at least in part on use of the electrosurgical electrode indicated by the tracking element.

13. The intelligent electrode device of claim 9, wherein the user feedback device issues an alert to a user of the electrosurgical electrode based at least in part on use of the electrosurgical electrode indicated by the tracking element.

14. The intelligent electrode device of claim 9, wherein the user feedback device issues an alert when the electrosurgical electrode is designated as a single-use needle and the electrosurgical electrode has already been used.

15. The intelligent electrode device of claim 1 further comprising a monopolar electrode tip.

16. The intelligent electrode device of claim 1 further comprising a bipolar electrode tip.

17. The intelligent electrode device of claim 1 further comprising a tungsten electrode tip having a radius of 50 microns or smaller.

18. A tracking, system comprising:
an intelligent electrode device having a tracking element to indicate use of an electrosurgical electrode;
a monitoring subsystem to receive data indicating use of the electrosurgical electrode from the tracking element; and
an alert delivery subsystem operatively associated with the monitoring subsystem to issue an alert based at least in part on use of the electrosurgical electrode indicated by the tracking element.

19. The tracking system of claim 8, wherein the alert delivery subsystem issues the alert to a manufacturer of the electrosurgical electrode.

20. The tracking system of claim 1 further comprising a user feedback device to issue an alert to a user of the intelligent electrode device based at least in part on use of the electrosurgical electrode indicated by the tracking element.

21. A method comprising:
automatically tracking use of an electrosurgical electrode in an intelligent electrode device;
indicating overuse of the electrosurgical electrode.

22. The method of claim 21 wherein indicating overuse is after a single use of the electrosurgical electrode.
